(19) United States
(12) Patent Application Publication
(10) Pub. No.: US 2002/0173034 A1
(43) Pub. Date: Nov. 21, 2002

(54) Centrifuge apparatus and methods for separating components from a cell culture device

(76) Inventors: Emilio Barbera-Guillem, Powell, OH (US); John M. Swartz, Westerville, OH (US)

Correspondence Address: BENESCH, FRIEDLANDER, COPLAN & ARONOFF LLP, ATTN: IP DEPARTMENT DOCKET CLERK, 2300 BP TOWER, 200 PUBLIC SQUARE, CLEVELAND, OH, 44114, US

(21) Appl. No.: 09/861,686

(22) Filed: May 21, 2001

(51) Int. Cl. C12N 5/00; C12M 1/00; C12N 5/02

(52) U.S. Cl. 435/325; 435/283.1

(57) ABSTRACT

An apparatus for use in a swinging bucket centrifuge rotor, the apparatus comprising a pair of sidewalls wherein each sidewall comprises a plurality of longitudinally spaced and laterally disposed partitions; wherein each pair of adjacent partitions defines a slot which is dimensioned to receive a cell culture device. The apparatus comprises a centrifuge bucket or an adaptor for use with a centrifuge bucket. Also provided is a method for harvesting one or more components of a cell culture contained within a cell culture device by centrifuging the cell culture device as supported by the apparatus during centrifugation, and then removing separately the one or more components of the cell culture from the centrifuged cell culture device. Further provided is a method for harvesting anchorage-dependent cells by distending the membrane surface to which the cells adhere in a process of loosening the cells, contacting and mixing the loosened cells with a fluid in forming a cell suspension, and then harvesting the anchorage-dependent cells by removing the cell suspension from the cell culture device.

### FIELD OF INVENTION

The present invention relates generally to harvesting of a cell culture; and more particularly to methods and centrifuge apparatuses for supporting one or more cell culture devices for centrifugation.

### BACKGROUND OF THE INVENTION

Centrifugation is typically used to separate components of cell cultures. Conventional containers for culturing cell culture devices include flasks and roller bottles. Thus, to harvest one or more components from such conventional containers, it is it is first necessary to open the container to allow for pipetting. In that regard, flasks and roller bottles have screw caps that require temporary removal to allow for pipetting of one or more of cell culture medium and cultured cells out of the cell culture container. In continuing the harvesting operation, transfer of the cell culture to an additional vessel (e.g., centrifuge tubes) for centrifugation is required. Thus, opening a flask or roller bottle, and transferring the one or more components into a vessel for centrifugation (one or more centrifuge tubes) is labor intensive, and necessitates an open system which greatly increases a breach in the maintenance of sterility of the cultures. Further, when anchorage-dependent cells are harvested from a cell culture, an enzyme solution (typically containing trypsin) is introduced into the cell culture to loosen the cell attachment from the surface to which they adhere. This is an extra step which is also labor intensive, and necessitates an open system thereby increasing a breach in the maintenance of sterility of the cultures.

Typically, the container or vessel to be centrifuged is place into a centrifuge carrier. The centrifuge carrier is commonly removable from (to facilitate cleaning or replacement), and pivotally attached to, a centrifuge rotor. Conventional centrifuge carriers include centrifuge buckets that hang detachably mounted to a centrifuge rotor so that they can swing from a depending vertical position to a horizontal position responsive to centrifugal forces. In these swinging bucket-type rotors, the centrifuge buckets are detachably mounted on pivot pins disposed on the outer arms of the rotor. When power is applied to the drive shaft of the centrifuge, the rotor arms are rotated, and each bucket swings outwardly until the axis thereof is perpendicular to the rotational axis of the rotor. However, existing centrifuge buckets have been adapted only for the centrifugation of tubes and microtiter plates.

Accordingly, there is a need to centrifuge a cell culture device to obtain one or more components without the need to compromise the maintenance of sterility of the culture and/or to transfer the culture to another container for centrifugation.

### SUMMARY OF THE INVENTION

In accordance with the present invention, these problems are solved by novel methods for separating one or more components of a cell culture contained in a novel cell culture device (see, e.g., Genetic Engineering News, vol. 20, No. 21, December 2000, “OptiCell™ Concept for Cell Culture Operations”, by the present assignee); and apparatuses developed to support one or more novel cell culture devices for centrifugation, wherein preferably each cell culture device contains a cell culture.

In one aspect of the present invention, provided is an apparatus for supporting one or more cell culture devices, wherein the apparatus comprises a novel adaptor that evenly distributes a number of cell culture devices across a standard centrifuge bucket into which is placed the adaptor. The adaptor may further comprise one or more handles which facilitate placing the adaptor into, and removing the adaptor from, a centrifuge bucket.

In another aspect of the present invention, provided is an apparatus for supporting one or more cell culture devices, wherein the apparatus comprises a novel centrifuge bucket comprising a plurality of partitions arranged within the chamber of the bucket, and wherein each pair of partitions defines a slot in which a cell culture device containing a cell culture may be placed in loading the cell culture device into the bucket. The centrifuge bucket further comprises sockets on its outer surface for allowing the bucket to be hung on the pivot pins of the centrifuge rotor.

In another aspect of the present invention, provided is a method for separating anchorage-dependent cells from adhering to a cell culture device, wherein the method comprises inflating the cell culture device with air in causing the one or more membrane surfaces, to which the cells are adhered, to expand and flex. Such expansion and flexing of the membrane causes adherent anchorage-dependent cells to loosen their attachment such that the cells may be further separated from the surface by agitation of the cell culture device containing the cell culture. The method may further comprise deflating the cell culture device (e.g., releasing the quantity of air used to inflate the device), centrifuging the cell culture device to pellet the cultured cells, and removing the component of interest from the centrifuged cell culture device.

Also provided is a method of separating components of cell cultures using the apparatus according to the present invention, wherein the method comprises placing a cell culture device into the apparatus (centrifuge bucket adapted to receive the cell culture device; or an adaptor, contained within a centrifuge bucket) adapted to receive and support the cell culture device; centrifuging the cell culture device contained in the apparatus; and recovering the component of interest from the centrifuged cell culture device.

The above and other objects, features, and advantages of the present invention will be apparent in the following Detailed Description of the Invention when read in conjunction with the accompanying drawings in which reference numerals denote the same or similar parts throughout the several illustrated views and embodiments.

### BRIEF DESCRIPTION OF THE DRAWINGS

FIG. 1 is a perspective view of one embodiment of the apparatus according to the present invention comprising an adaptor for supporting one or more cell culture devices.

FIG. 2 is a perspective view of another embodiment of the apparatus according to the present invention comprising an adaptor for supporting one or more cell culture devices.

FIG. 3 is a perspective view of another embodiment of the apparatus according to the present invention comprising an adaptor for supporting one or more cell culture devices.

FIG. 4 is a perspective view of another embodiment of the apparatus according to the present invention comprising a centrifuge bucket for supporting one or more cell culture devices.

FIG. 5 is a perspective view of the apparatus according to the present invention comprising a centrifuge bucket for supporting one or more cell culture devices.

FIG. 6 is a perspective view of the apparatus according to the present invention comprising a centrifuge bucket for supporting one or more cell culture devices, wherein the bucket is hung detachably mounted on a centrifuge rotor.

FIG. 7 is a side view of a cell culture device containing air in a sufficient amount to expand the membrane surface to which anchorage-dependent cells adhere in a method of detaching the cells from the membrane surface.

### DETAILED DESCRIPTION OF THE INVENTION

Definitions:

Throughout the specification of the application, various terms are used such as “top”, “bottom”, “inward”, “outward”, “upper”, “lower”, and the like. These terms are words of convenience in order to distinguish between different elements. While such terms are provided to explain the apparatus relative to positions in which the apparatus may normally be used, such terms are not intended to be limiting as to how the different elements may be utilized.

The term “cells” is used herein, for the purposes of the specification and claims, to mean one or more of live cells, cells comprising cellular aggregates, or an organized structure or network of cells in forming a tissue, as apparent to those skilled in the art. Cells typically cultured are known to those skilled in the art to include, but are not limited to, cell lines, tumor cells, hematopoietic cells, cells isolated from a tissue, genetically engineered cells, animal cells, insect cells, mammalian cells, human cells, transgenic cells, transformed cells, transfected cells, or other cell type desired to be cultured. Cellular aggregates may be comprised of a single cell type or of multiple cell types. Tissue may be exemplified by, but not limited to, one or more tissue fragments that may be introduced into the device according to the present invention, or systematic introduction of cells of various cell types needed to form a tissue, using standard techniques known in the art (e.g., culturing cells on a three dimensional synthetic (e.g., polyglycolic acid) or natural (e.g., collagen or extra-cellular) matrix).

The term “tissue culture medium” is used herein, for the purposes of the specification and claims, to mean a liquid solution which is used to provide sufficient nutrients (e.g., vitamins, amino acids, essential nutrients, salts, and the like) and properties (e.g., osmolarity, buffering) to maintain living cells (or living cells in a tissue) and support their growth. Commercially available tissue culture medium is known to those skilled in the art. The term “cell culture medium” is used herein, for the purposes of the specification and claims, to mean tissue culture medium that has been incubated with cultured cells in forming a cell culture; and more preferably refers to tissue culture medium that further comprises substances secreted, excreted or released by cultured cells, or other compositional and/or physical changes that occur in the medium resulting from culturing the cells in the presence of tissue culture medium.

The term “dissociation reagent” is used herein, for the purposes of the specification and claims, to mean a solution or fluid which is contacted with anchorage-dependent cells and causes the cells to dissociate (loosen their cell attachment, and may become detached) from the surface to which they adhere. Such solutions are well known in the art to include, but are not limited to, a solution comprising one or more chelators (ethylenediamine tetraacetate, “EDTA”; ethylene glycol-bis beta-aminoethyl ether N,N,N′,N′-tetraacetic acid, “EGTA”; versen; and the like), one or more proteolytic enzymes (e.g., ficin, pepsin, trypsin, chymotrypsin, papain, and the like, with trypsin being a preferred enzyme), or a combination thereof (e.g., a combination of trypsin and EDTA). Dissociation reagents are available commercially, or can be prepared using methods and formulations well known in the art.

Provided are apparatuses and methods that may be used for separating one or more components of a cell culture contained in a cell culture device, wherein the term “cell culture device” is used herein for purposes of the specification and claims to mean a cell culture device with the following preferred characteristics. The cell culture device comprises a frame; at least one gas permeable, liquid impermeable membrane (and preferably two gas permeable, liquid impermeable membranes) extended and stretched taut over and securedly sealed to the frame in forming a culture chamber; and at least one resealable aperture through the frame which allows substances to be introduced into, or withdrawn from, the culture chamber. Alternatively, the frame may comprise a gas permeable membrane on one side, and an opposite side comprises a hard plastic surface typical of standard tissue culture containers. In a preferred embodiment, the cell culture device comprises a frame sandwiched between 2 gas permeable membranes in forming at least one culture chamber therebetween. The frame is sufficiently rigid to provide a housing for assembling the cell culture device. The at least one gas permeable membrane is of suitable thickness to provide sufficient gas permeability to accommodate cell growth in the chamber, and to provide sufficient structural integrity for handling the apparatus. Further, the one or more membranes are of a sufficient optical transparency and clarity so as to observe during culture, the color of the tissue culture medium, and cellular characteristics (e.g., growth and morphology of cells such as by microscopy). The frame has at least one resealable aperture, and preferably at least two resealable apertures, which allows substances to be introduced into, or withdrawn from, the culture chamber. Each aperture of the frame may serve as a passageway into which is guided a portion of an instrument (e.g., needle or pipette or pipette tip) for introducing a substance into or withdrawing a substance from the culture chamber. The dimensions of the cell culture device may depend on one or more factors including, but not limited to, the desired fluid capacity of the culture chamber formed therewith, and the dimensions of the culture chamber. In a preferred embodiment, the cell culture device is generally rectangular in shape, and comprises a dimension sufficient to be accommodated, and be substantially held in position, by a standard mechanical stage specimen holder (e.g., that accommodates a 96 well microtiter plate) of a microscope. In a more preferred embodiment, the cell culture device has a length in a range of from about 10 cm to about 13.5 cm, a width in a range of from about 7 cm to about 9 cm, and a height in a range of from about 0.2 cm to about 1.0 cm. In a most preferred embodiment, the cell culture device has a length of about 12.7 cm, a width of about 8.5 cm, and a height of about 0.58 cm.

Now referring to the drawings in general, it is apparent that apparatus for supporting the aforementioned cell culture device (preferably containing a cell culture), in accordance with the present invention, generally comprises a housing. The detailed relationship between the individual features of the apparatus is not critical to the invention, insofar as to whether they be manufactured as separate components which are then assembled into the unit comprising the apparatus or as an integral one-piece unit such as by a molding process. Therefore, it is apparent to one skilled in the art that the apparatus according to the present invention may be fabricated with methods now known in the art, or later devised, without departing from the spirit or scope of the invention. Additionally, the apparatus may be formed from a material which provides the requisite rigidity and support for holding a plurality of cell culture devices therein in typical conditions encountered in centrifugation (particularly, the relatively low gravitational (g) forces typically used to pellet cultured cells as standard in the art). As the specific character of the material does not in and of itself constitute the subject matter of the present invention, it should be apparent to those skilled in the art that a wide latitude of choice may be exercised in selecting a material suitable for formation and/or fabrication of the apparatus. Typical materials may include, but are not limited to, metal, synthetic resinous material, natural rubbers or polymeric materials, and the like. In a preferred embodiment, the apparatus may be generally molded from synthetic resinous material, such as plastic; and in a more preferred embodiment, the material comprises polyethylene or polypropylene.

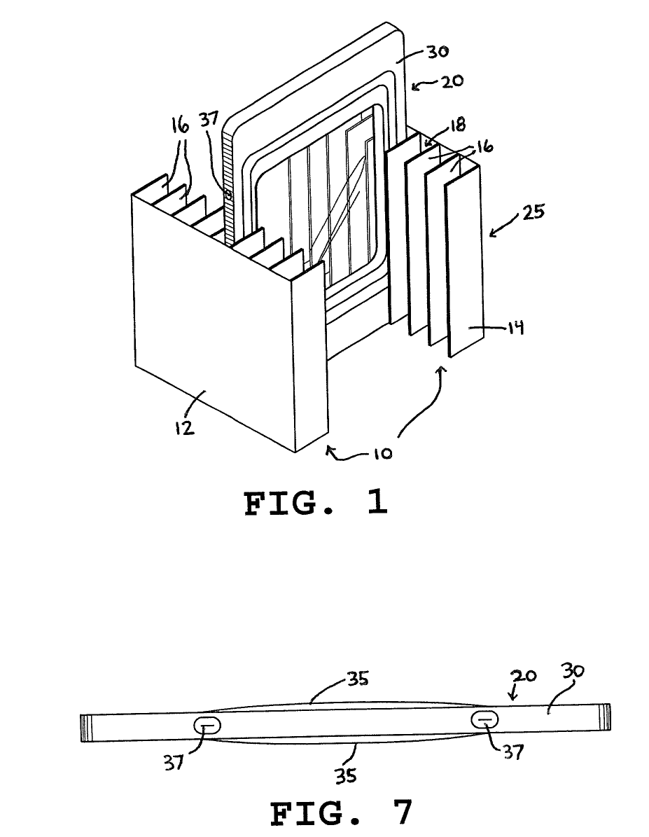

Referring particularly to FIGS. 1-3, in one embodiment of the present invention, provided is an apparatus for supporting one or more cell culture devices, wherein apparatus 10 comprises adaptor 10 for use in a centrifuge bucket for a swinging bucket centrifuge rotor. Commercially available centrifuge buckets vary in size and shape; however, typically a centrifuge bucket has an interior having a polygonal shape that is used for receiving an centrifuge tube adaptor that evenly distributes and supports one of a plurality of sizes and shapes of centrifuge tubes in the bucket for centrifugation. Thus, the shape of adaptor 10 is not particularly critical, and is generally chosen to conform to the inner shape of the centrifuge bucket into which the adaptor is inserted. Thus, as shown in FIGS. 1-3 adaptor 10 may comprise a polygonal shape to support one or more cell culture devices, as well as to conform to the inner shape of the centrifuge bucket into which the adaptor is inserted. More particularly, the dimensions of adaptor 10 are such that the adaptor can be inserted into, and snugly fit inside, the centrifuge bucket. With reference to FIG. 1, in one embodiment, adaptor 10 comprises a housing formed by two sections or sidewalls 12, 14 that are adapted to, in combination, secure one or more cell culture devices 20. Each of sidewalls 12, 14 comprises a plurality of longitudinally spaced and laterally disposed partitions 16. Thus, as illustrated in FIG. 1, when the two sidewalls 12, 14 are arranged so that they are positioned in parallel, spaced apart relation, formed is a plurality of slots 18. Preferably, each slot 18 is dimensioned to receive a cell culture device in a snug fit to facilitate supporting the cell culture device in a manner sufficient to be used in a centrifugation process. A cell culture device may be manually loaded into each slot 18. In a process of using adaptor 10 for supporting one or more cell culture devices 20, sidewalls 12, 14 of adaptor 10 may be first inserted into, and positioned within, the interior of the centrifuge bucket so as to receive the one or more cell culture devices 20. Each of the one or more cell culture devices 20 to be supported by adaptor 10 may then be inserted into a slot 18. Alternatively, sidewalls 12, 14 are positioned in parallel, spaced apart relation, and each of the one or more cell culture devices 20 (desired to be supported by adaptor 10) may be inserted into a slot 18 in forming assembly 25. Assembly 25 may then be inserted into the centrifuge bucket.

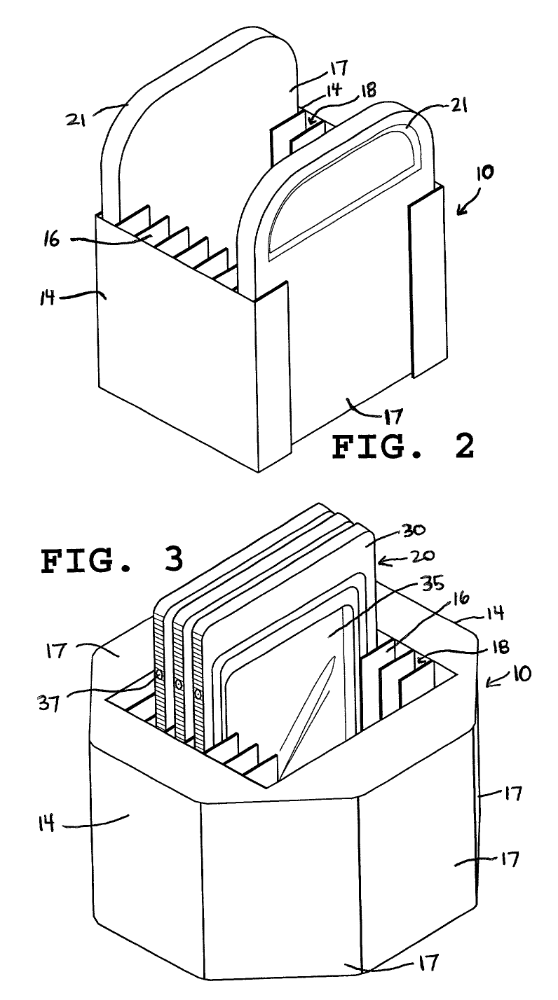

With reference to FIGS. 2 & 3, in another embodiment, adaptor 10 comprises a housing, formed as an integral unit, for supporting one or more cell culture devices 20; wherein the housing comprises a pair of laterally spaced sidewalls 14 which are interconnected by endwalls 17 and a bottom, in forming an interior chamber for accommodating one or more cell culture devices. A plurality of longitudinally spaced and laterally disposed partitions 16 are provided on sidewalls 14 of the housing of adaptor 10, for purposes of maintaining separation of adjacent cell cultures when a plurality of cell culture devices is inserted into adaptor 10. More particularly, each pair of adjacent partitions defines a slot 18 in which may be placed and supported a cell culture device 20. Preferably, each slot 18 is dimensioned to receive a cell culture device in a snug fit to facilitate supporting the cell culture device in a manner sufficient to be used in a centrifugation process. In a process of using adaptor 10 for supporting one or more cell culture devices 20, adaptor 10 may be first inserted into, and positioned within, the interior of the centrifuge bucket so as to receive the one or more cell culture devices 20. Each of the one or more cell culture devices 20 to be supported by adaptor 10 may then be inserted into a slot 18. Alternatively, each of the one or more cell culture devices 20, desired to be supported by adaptor 10, may be inserted into a slot 18 of adaptor 10, and then adaptor 10 may be inserted into the centrifuge bucket. As illustrated in FIG. 2, adaptor 10 may further a plurality of handles 21, and more preferably a pair of handles 21, which may facilitate lifting adaptor 10, and inserting adaptor 10 into a centrifuge bucket.

With reference to FIGS. 4-6, the apparatus according to the present invention comprises centrifuge bucket 10 which may be mounted on a swinging bucket centrifuge rotor by any means known in the art that may include, but is not limited to, a hinge or pins, in allowing the centrifuge bucket to pivot about a single axis in response to the forces of centrifugation. Typically, the centrifuge rotor comprises pins onto which the centrifuge bucket is hung via grooves located on the sides of the centrifuge bucket for engaging the pins. However, other arrangements are known in the art; e.g., the centrifuge bucket may comprise the pins, and the rotor may have grooves for engaging the pins. Centrifuge rotor 40 is provided with arms 42 which extend radially outward from the axis of rotor 40. Arms 42 terminate in support portions 44 which support first ends of pins 46, the other ends of which are connected to opposite sides of centrifuge bucket 10. More particularly, centrifuge bucket 10 comprises two sockets 48, each disposed on opposite sides of the exterior of centrifuge bucket 10, the dimensions of sockets 48 being such that sockets 48 can engage pins 46. In this arrangement, centrifuge bucket 10 is suspended (e.g., hung) on ends of pins 46, between arms 42, in providing a means by which centrifuge bucket 10 may pivot (e.g., swing) in response to forces of centrifugation. Centrifuge bucket 10 comprises a housing, formed as an integral unit, for supporting one or more cell culture devices 20. The housing of centrifuge bucket 10 comprises a pair of laterally spaced sidewalls 14 which are interconnected by endwalls 17 and a bottom 12, in forming an interior chamber for accommodating one or more cell culture devices. A plurality of longitudinally spaced and laterally disposed partitions 16 are provided on sidewalls 14 of the housing of centrifuge bucket 10, for purposes of maintaining separation of adjacent cell cultures when a plurality of cell culture devices is inserted into centrifuge bucket 10. More particularly, each pair of adjacent partitions defines a slot 18 in which may be placed and supported a cell culture device 20. Preferably, each slot 18 is dimensioned to receive a cell culture device in a snug fit to facilitate supporting the cell culture device in a manner sufficient to be used in a centrifugation process. In a process of using centrifuge bucket 10 for supporting one or more cell culture devices 20, centrifuge bucket 10 may be first hung on pins 46 of centrifuge rotor 40. Each of the one or more cell culture devices 20 to be supported by centrifuge bucket 10 may then be inserted into a slot 18. Alternatively, each of the one or more cell culture devices 20, desired to be supported by centrifuge bucket 10, may be inserted into a slot 18 of centrifuge bucket 10, and then centrifuge bucket 10 may be hung on centrifuge rotor 40, as previously described herein.

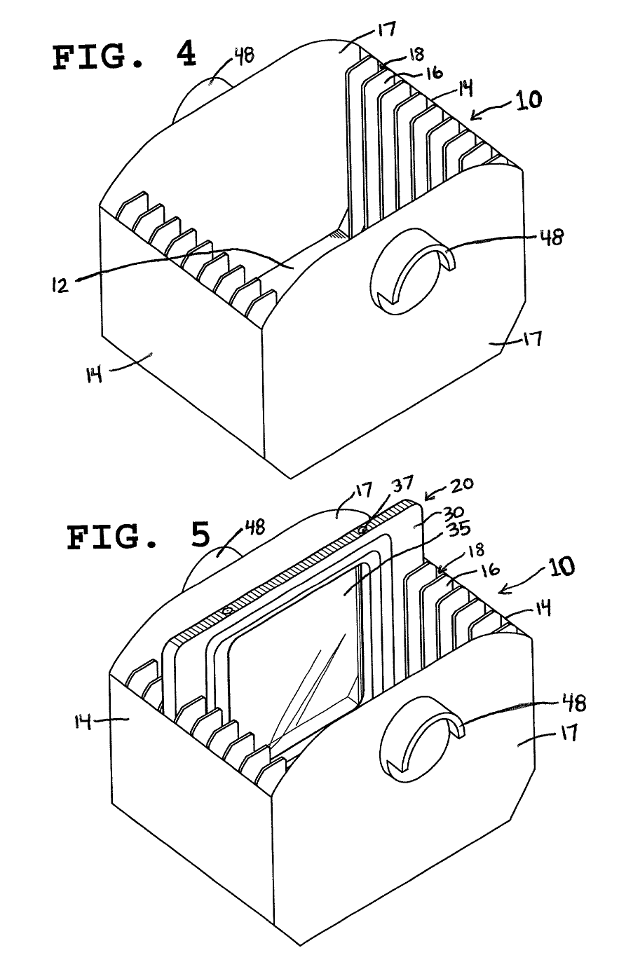

In accordance with the present invention, the apparatus for supporting one or more cell culture devices is selected from the group consisting of an adaptor for a centrifuge bucket, and a centrifuge bucket. In a preferred embodiment, the one or more cell culture devices 20 are loaded into the apparatus 10 so that cells pelleted by forces of centrifugation will collect in the chamber of the cell culture device 20 substantially along frame 30 rather than along a membrane 35 of the cell culture device 20. Further, it is preferable that a cell culture device be loaded so that access ports 37 of cell culture device 20 will not be adjacent to the bottom of the centrifuge bucket (e.g., access ports 37 of a loaded cell culture device 20 will be facing sidewalls (as illustrated in FIGS. 1 & 3) or the opening (as illustrated in FIGS. 5 & 6) of the apparatus). It will be apparent to one skilled in the art that the optimal number of cell culture devices which may be loaded into the apparatus according to the present invention depends on factors that may include, but are not limited to, the size of the apparatus, desired size of centrifuge bucket to be used, the desired size of the cell culture device, and the size of the centrifuge rotor (e.g., number of arms, etc.). Thus, it is apparent to one skilled in the art that different sizes of the apparatus according to the present invention and/or cell culture devices may require different shapes and spacings (e.g., size of slots, etc.) and patterns, and that the present invention is not limited to the shapes and spacings and patterns illustrated.

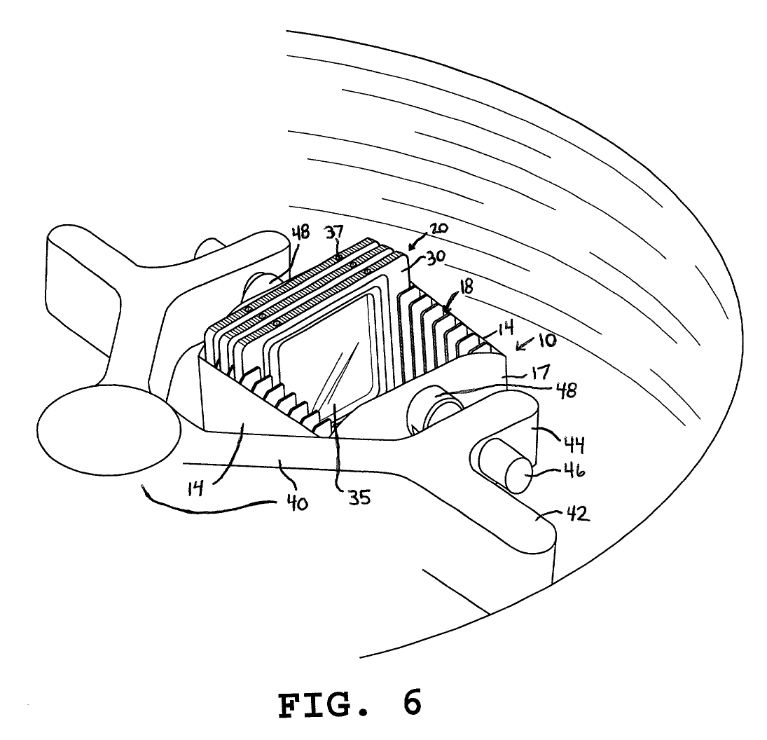

In accordance with the present invention, provided is a method of centrifuging one or more cell culture devices, the method comprising: loading the one or more cell culture devices into a centrifuge, wherein each of the one or more cell culture devices is supported in a slot being formed by a pair of adjacent partitions, a slot being dimensioned to receive a cell culture device, wherein a plurality of slots are formed in an apparatus comprising a centrifuge bucket, and wherein a plurality of centrifuge buckets are mounted on a rotor in the centrifuge; operating the centrifuge; and removing the one or more cell culture devices from the centrifuge. Referring to FIG. 6, shown is a portion of the chamber of a centrifuge and a portion of a centrifuge rotor 40. In operation, the one or more cell culture devices to be centrifuged are loaded into the apparatus according to the present invention using one of the methods previously described herein in more detail. When using the method to centrifuge a single cell culture device, the cell culture device is supported in one centrifuge bucket, with appropriate balancing of weight on the rotor to be accomplished using methods standard in the art and before operating the centrifuge. Whereas using the method to centrifuge a plurality of cell culture devices, and depending on the number of cell culture devices comprising the plurality, the cell culture devices may be supported in one centrifuge bucket or may be supported by a plurality of centrifuge buckets, with the appropriate balancing of weight on the rotor to be accomplished using methods standard in the art and before operating the centrifuge. In accordance to the method, the apparatus comprises a centrifuge bucket, wherein the centrifuge bucket contains an adaptor according to the present invention inserted within a centrifuge bucket, or comprises a centrifuge bucket according to the present invention. Thus, as previously described herein in more detail, and depending on the embodiment, either the adaptor or the centrifuge bucket comprises a plurality of longitudinally spaced and laterally disposed partitions provided on sidewalls thereof, wherein each pair of adjacent partitions defines a slot dimensioned to receive and support a cell culture device. As illustrated in FIG. 6, with the one or more cell culture devices loaded into the centrifuge, the rotor and apparatus according to the present invention are in a resting position such that the centrifuge buckets, mounted on the rotor, hang vertically down under the influence of gravity.

Once loaded, the centrifuge is operated by initiating centrifugation for a controlled (predetermined) time and operational speed. As apparent to one skilled in the art, the time and operational speed may vary depending on the type of centrifuge used, size of the swinging bucket centrifuge rotor, type of cells to be pelleted (cells being contained in the one or more cell culture devices being centrifuged), and other factors. As an illustrative example, using a standard swinging bucket rotor (typically having a maximum rotational speed in a range of from about 2,000 rpm to about 5,000 rpm, wherein rpm is revolutions per minute), a preferred rotational speed in the operation of the centrifuge in the method according to the present invention may be a speed selected in a range of from about 1,000 rpm to about 2,000 rpm; and the length of time of centrifugation in operation of the centrifuge in the method according to the present invention may be a time selected in a range of from about 5 minutes to about 20 minutes. Upon rotation of the rotor during operation of the centrifuge, the mounted centrifuge buckets swing outwardly in response to centrifugal force to a horizontal position (e.g., bottom surface of each centrifuge bucket is in a radially outermost position). As will be described in more detail herein, during operation of the centrifuge, the centrifugal forces will then pellet cells (whether the cells are anchorage-independent or anchorage-dependent), that may be contained in each of the one or more cell culture devices, substantially against an inner surface of the frame of the cell culture device which is in a radially outermost position. When the rotor stops, the centrifuge buckets return to position of hanging vertically downward, and the centrifuged cell culture devices may then be removed from the centrifuge (e.g., lifting each of the one or more cell culture devices from the slot which supports it).

With the aforementioned description of a method for centrifuging one or more cell culture devices, it is also within the scope of the present invention to provide a method for separating components of a cell culture within a cell culture device using the apparatus according to the present invention. As previously eluded to herein, essentially the only available methods to harvest anchorage-dependent cells from a cell culture contained within a conventional cell culture apparatus (e.g., tissue culture flask, roller bottle or tissue culture plate) are: (a) to scrape the cells from the surface to which they adhere; or (b) treat the cells in the cell culture apparatus with a solution comprising a dissociation reagent introduced into the cell culture device to contact the cells and cause the cells to dissociate (loosen their cell attachment, and may become detached) from the surface to which they adhere. The detached cells are then collected from the cell culture apparatus. Either method causes trauma to the cells being harvested. For example, treatment of cultured cells with a dissociation reagent comprising a chelator (e.g., EDTA), or scraping the cells, can result in a decrease in viability. Treatment of cultured cells with a dissociation reagent comprising a proteolytic enzyme can degrade cell surface proteins, decrease cell viability, and interfere with a cell's biological activity. Such treatment also comprises extra steps which are labor intensive, and necessitates an open system thereby increasing a breach in the maintenance of sterility of the cultures. Additionally, once the cells are detached from the surface, or in harvesting anchorage-independent cells, the cells are typically transferred to a centrifuge tube for pelleting the cells by centrifugation; an extra step in handling of the cells.

In development of the present invention, it was found that using a cell culture device comprising a frame sandwiched between 2 liquid impermeable membranes (wherein at least one of the membranes is gas permeable), and wherein one or both membranes is used as a surface to which anchorage-dependent cells adhere in culturing the cells, centrifugation of the cell culture device can result in sufficient forces to detach the cells from adhering to the membrane surface. Thus, centrifugation may be used in a method to separate one or more components of a cell culture contained within a cell culture device and for harvesting the one or more components. More specifically, in using an apparatus according to the present invention, centrifugation at a preferred operational speed (e.g., from about 1,000 rpm to about 2,000 rpm) provides shear force sufficient to cause anchorage-dependent cells to loosen and detach from the membrane surface to which they adhere. Accordingly, centrifugation of the cell culture device may be used in a method of harvesting anchorage-dependent cells, in obviating the need to add a dissociation reagent to loosen anchorage-dependent cells. However, depending on factors such as the cell type comprising the anchorage-dependent cells, and the nature of the membrane surface to which they adhere, some cultured cells may have an attachment to a surface wherein some dissociation may facilitate harvesting the cells by centrifugation. Thus, in a method of centrifuging anchorage-dependent cells to harvest cells according to the present invention, the method may further comprise a pretreatment (as a step before the cell culture device is loaded in the centrifuge) of the cultured cells to be harvested, wherein the pretreatment comprises contacting the anchorage-dependent, cultured cells with a solution comprising a dissociation reagent, distending the membrane surface to which the cells adhere to loosen their attachment to the membrane surface (as describe in more detail herein), or a combination thereof (contact with a solution comprising a dissociation reagent and distending the membrane surface).

One or more cell culture devices containing anchorage-dependent cells may be centrifuged, as previously described herein in more detail, to cause the cells to pellet within each cell culture device centrifuged. Similarly, one or more cell culture devices containing anchorage-independent cells may be centrifuged to cause the cells to pellet within each cell culture device centrifuged. Each cell culture device 20 to be centrifuged is loaded into apparatus 10 by placing an edge of frame 30 so that it is adjacent to the bottom of the centrifuge bucket; i.e., loaded so that cells pelleted by forces of centrifugation will collect substantially along an inner surface of frame 30 of each cell culture device 20 rather than along a membrane 35 of each cell culture device 20 being centrifuged (see, e.g., FIGS. 1, 3, 5, 6). In such an arrangement, when the rotor is in a resting position, one or more membranes 35 of each cell culture device 20 are in a vertical plane; and during the operating speed of centrifugation, the one or more membranes 35 of each cell culture device 20 are in a horizontal plane. When a cell culture device is in a horizontal plane, the centrifugal forces pull anchorage-dependent cells away from the membrane and toward an inner surface of the frame which is in the cell culture chamber of the cell culture device, and which is in a radially outermost position (e.g., the location depending on how the cell culture device is loaded within the apparatus). Thus, centrifugal force alone may be sufficient to pellet cells within a cell culture device. As previously mentioned herein, the cultured cells may be pretreated in the cell culture device before centrifugation so as to facilitate harvesting of the cells by centrifugation. Alternatively, when a cell culture device is in a horizontal plane, the centrifugal pulls anchorage-independent cells out from being suspended in the cell culture medium and toward an inner surface of the frame which is in the cell culture chamber of the cell culture device, and which is in a radially outermost position (e.g., the location depending on how the cell culture device is loaded within the apparatus).

After centrifugation, one or more components may be separated from each cell culture device centrifuged. For example, each cell culture device centrifuged may be removed from the centrifuge, and a component comprising cell culture medium may be separated from the pelleted cells contained with the cell culture device by removing the supernatant comprising cell culture medium from the cell culture device in harvesting a component comprising cell culture medium. For example, the cell culture medium to be harvested may be aspirated through one of the access ports 37 (e.g., inserting a syringe with a blunt tip, or inserting a pipette tip, into the access port, and withdrawing the supernatant comprising cell culture medium), and out of the cell culture device in harvesting a component comprising cell culture medium. By aspirating substantially all of the supernatant (comprising cell culture medium) from the cell culture device, the pelleted cells remaining in the cell culture device comprise a separated component. The method according to the present invention, may further comprise harvesting the cells remaining in the cell culture device by introducing a fluid (e.g., tissue culture medium, or a physiological solution (e.g., buffer or saline solution), or the like, known in the art to be biocompatible with cultured cells) into the cell culture device to resuspend the pelleted cells (which may be facilitated by agitating the cell culture device in contacting and mixing the cells with the fluid contained therein) in forming a cell suspension, and then aspirating (or otherwise removing) the cell suspension out of the cell culture device, in harvesting the cells.

Anchorage-dependent cells may be harvested by another method according to the present invention. In this example, cultured cells comprised of anchorage-dependent cells are harvested from a cell culture device, wherein the cell culture device comprises at least one gas permeable, liquid impermeable membrane that comprises a surface to which anchorage-dependent cells adhere. Each membrane, of the at least one membrane, is elastomeric. Therefore, each membrane is expandable upon contact with a substance present in a sufficient amount to pressure the membrane to distend. Because the membrane is elastomeric, it is biased to return to a less distended shape (e.g., to a preformed shape or non-distended state or a less expanded state) after removal of a sufficient amount of the substance to reduce pressure that causes distension of the membrane. The method for harvesting the anchorage-dependent cells from a cell culture contained within a cell culture device comprises: injecting a substance selected from the group consisting of air, fluid, and a combination thereof into the cell culture device, wherein the substance is injected in an amount sufficient to distend the at least one membrane and cause the anchorage-dependent cells to loosen their attachment from the membrane surface to which they are attached; withdrawing the substance in an amount sufficient for the at least one membrane to return to a less distended shape; contacting and mixing the loosened cells with a fluid within the cell culture device in causing the cells to detach from the membrane surface and to form a suspension of cells; and removing the suspension of cells from the cell culture device in harvesting the anchorage-dependent cells. In a preferred embodiment of this method of harvesting anchorage-dependent cells from a cell culture, the two step process (comprised of introducing a substance into the cell culture device to cause distension of the at least one membrane, and withdrawing the substance in an amount to return the at least one membrane to a less distended shape) is repeated multiple times; and more preferably is repeated a number of times in a range of from about 2 to about 5. Depending on factors such as the cell type comprising the anchorage-dependent cells, and the nature of the membrane surface to which they adhere, some cultured cells may have an attachment to a surface wherein some dissociation may facilitate harvesting the cells by a method in which the membrane surface is distended. Thus, in another embodiment of a method of harvesting anchorage-dependent cells, the substance which may be introduced to distend the membrane surface may comprise a fluid comprising a dissociation reagent. Because distension of the membrane surface also loosens cell attachment, the concentration of a dissociation reagent which is added to the cultured cells in the cell culture device is typically much less than that used in conventional cell culture apparatuses. For example, where a dissociation reagent comprising trypsin in a concentration of from about 10% to about 25% may typically be used to harvest cells from a conventional cell culture apparatus, a dissociation reagent comprised of about 2.5% trypsin may be used in the method of harvesting anchorage-dependent cells according to the present invention. Using a relatively smaller amount of a dissociation reagent may minimize trauma to the cells associated with contacted with the dissociation reagent.

As previously described herein, a cell culture device 20 comprises at least one membrane; e.g., may comprise a frame 30 sandwiched between 2 liquid impermeable membranes 35, or one membrane 35 and a opposing hard plastic surface (wherein at least one membranes is gas permeable). The at least one membrane, which may be used as a surface to which anchorage-dependent cells adhere in culturing the cells,: (a) is expandable upon introduction, into the cell culture device, of a substance (comprising air, fluid, or a combination thereof) in an amount sufficient to cause distension of the at least one membrane of the cell culture device; and (b) when distended, anchorage-dependent cells loosen their attachment. Contacting and mixing the loosened cells with a fluid results in the loosened cells to become detached from the membrane surface and then released into the fluid in forming a cell suspension. In illustrating this method, injecting (e.g., through a resealable access port 37 using a syringe and blunt tip) a substance into a cell culture device 20 can result in the one or more membranes 35, to which anchorage-dependent cells are adhered, to distend. Distension of a membrane may result in sufficient forces to stretch the means by which the anchorage-dependent cells adhere to the membrane in causing the cells to loosen from the distended membrane surface. Repeatedly distending and allowing the membrane surface to return to a less distended shape may further facilitate the cells to loosen their attachment to the membrane surface. Once the cells are loosened, the cells are contacted and mixed (e.g., by agitation of the cell culture device or by a pipetting action) with a fluid, which may be introduced into the cell culture device or may already be present in the cell culture device, in causing the cells to detach from the surface and be suspended in a fluid (in forming a cell suspension). As previously described herein in more detail, the fluid may further comprise a dissociation reagent. The cells may then be harvested by removing (e.g., by aspirating) the cell suspension from the cell culture device, or by centrifuging the cell culture device in causing the cells to pellet and then harvesting the cells (as previously described herein in more detail). Thus, the method of harvesting the anchorage-dependent cells, by distending the membrane surface to which they adhere, may further comprise (as a step following the distension of the membrane) centrifuging the cell culture device.

In one illustrative example, a cell culture device contains a cell culture comprising cell culture medium, and anchorage-dependent cells adhered to a membrane surface of the cell culture device. A substance (e.g., comprising air, fluid, or a combination thereof) may be injected into cell culture device 20, the amount of the substance being sufficient to cause the one or more membranes 35 of cell culture device 20 to expand, as illustrated in FIG. 7. It is noted that if the cell culture device comprises two membrane surfaces, either or both of the surfaces may be used as a surface for attachment of anchorage-dependent cells (e.g., by seeding one side with cells, flipping the device over, and seeding the other side with cells). Thus, this method of harvesting anchorage-dependent cells may be from one or more membrane surfaces of the cell culture device. As apparent to one skilled in the art, the amount of substance sufficient to expand the one or more membrane surfaces of the cell culture device will depend on factors which include, but are not limited to, the size of the cell culture device utilized, fluid capacity of the chamber of the cell culture device, and the thickness of the membranes. For example, in a preferred embodiment, the cell culture device comprises a length of about 12.7 cm, a width of about 8.5 cm, and a height of about 0.58 cm (having a cell culture chamber of a length of about 7.4 cm, a width of about 6.4 cm, and a height of about 2 cm); and comprises a membrane thickness of from about 2 mil to about 4 mil. An amount of a substance sufficient to expand the one or membranes of a preferred cell culture device may be a total volume in a range of from about 20 ml to about 30 ml (e.g., about 10 ml of fluid and a volume of air corresponding to about 20 ml; or about 30 ml of fluid; or about 14 ml of fluid and a volume of air corresponding to about 14 ml). Depending on the cell type, the anchorage-dependent cells may become loosened from the expanded membrane surface within seconds to minutes after the membrane surface expands. For example, in continuing with this preferred embodiment, anchorage-dependent cells comprising a cancer cell line were cultured in 10 ml of tissue culture medium in the cell culture device for 24-36 hours to ensure cell attachment onto a membrane surface. In one example, the cells were loosened by injection and aspiration of a substance comprising a fluid (e.g., either PBS, or a buffered solution containing 2.5% trypsin and/or 1 mM EDTA). In this example, injected into the cell culture device was an additional 20 ml of the fluid (thereby expanding the membranes with a total volume of about 30 ml), about 20 ml of medium was then aspirated from the cell culture device, and this two-step process was repeated twice more. In one illustration, the membrane was distended twice with a fluid that lacked the dissociation reagent, and then the fluid comprising the dissociation reagent was used to distend the membrane a third time. In either case, the cell culture device was then gently agitated, and substantially all the anchorage-dependent cells became detached and then suspended in the medium remaining in the cell culture device in forming a cell suspension. The cell suspension was then removed from the cell culture device, such as being aspirated through an access port as previously described herein in more detail, in harvesting the anchorage-dependent cells from the cell culture device. In another example, and in continuing with this preferred embodiment, anchorage-dependent cells comprising a cancer cell line were cultured in 10 ml of tissue culture medium in the cell culture device for 24-36 hours to ensure cell attachment onto a membrane surface. In this example, the cells were loosened by repeated injection and aspiration of a substance comprising air. In this example, a syringe was used to inject a volume of air comprising 20 ml into the cell culture device (thereby expanding the membranes with a total volume of about 30 ml of a combination of air and fluid), about 20 ml of air was then aspirated from the cell culture device, and this two-step process was repeated twice more. As previously described herein in more detail, in repeating the two-step process, the fluid (present in the cell culture device) may further comprise a dissociation reagent. The cell culture device was then gently agitated, and substantially all the anchorage-dependent cells became detached and then suspended in the medium remaining in the cell culture device in forming a cell suspension. The cell suspension was then removed from the cell culture device, such as being aspirated through an access port as previously described herein in more detail, in harvesting the anchorage-dependent cells from the cell culture device. As noted previously, this method may further comprise centrifuging the cell device (e.g., as comprising the step of agitating the cell culture device or as an additional step following agitation of the cell culture device) in a step before the suspension of cells is removed from the cell culture device.

The foregoing description of the specific embodiments of the present invention have been described in detail for purposes of illustration. In view of the descriptions and illustrations, others skilled in the art can, by applying, current knowledge, readily modify and/or adapt the present invention for various applications without departing from the basic concept, and therefore such modifications and/or adaptations are intended to be within the meaning and scope of the appended claims.

What is claimed is:

1. An apparatus for use in a swinging bucket centrifuge rotor, the apparatus comprising: a pair of sidewalls wherein each sidewall comprises a plurality of longitudinally spaced and laterally disposed partitions; wherein each pair of adjacent partitions defines a slot which is dimensioned to receive a cell culture device.

2. The apparatus according to claim 1, wherein the apparatus comprises an adaptor for use in a centrifuge bucket.

3. The apparatus according to claim 2, wherein the adaptor comprises two sidewalls which, when positioned in parallel spaced apart relation, form a plurality of slots with each slot being dimensioned to receive, and into which may be placed and supported, a cell culture device.

4. The apparatus according to claim 2, wherein the adaptor comprises:
two sidewalls which, when positioned in parallel spaced apart relation, form a plurality of slots with each slot being dimensioned to receive, and into which may be placed and supported, a cell culture device,
a plurality of endwalls, and
a bottom,
in forming an interior chamber for accommodating one or more cell culture devices.

5. The apparatus according to claim 2, wherein the adaptor further comprises a plurality of handles.

6. A method for supporting one or more cell culture devices in the apparatus according to claim 2, the method comprising the steps of:
inserting the adaptor into a centrifuge bucket; and
inserting each cell culture device, of the one or more cell culture devices to be supported, into a slot of the adaptor;
in supporting the one or more cell culture devices.

7. A method for supporting one or more cell culture devices in the apparatus according to claim 2, the method comprising the steps of:
inserting each cell culture device, of the one or more cell culture devices to be supported, into a slot of the adaptor; and
inserting the adaptor into a centrifuge bucket;
in supporting the one or more cell culture devices.

8. The apparatus according to claim 1, wherein the apparatus comprises a centrifuge bucket.

9. The apparatus according to claim 8, wherein the centrifuge bucket comprises:
two sidewalls which, when positioned in parallel spaced apart relation, form a plurality of slots with each slot being dimensioned to receive, and into which may be placed and supported, a cell culture device,
a plurality of endwalls, and
a bottom,
in forming an interior chamber for accommodating one or more cell culture devices.

10. The apparatus according to claim 8, wherein the centrifuge bucket further comprises two sockets disposed on opposite sides of the exterior of centrifuge bucket, and wherein the sockets are dimensioned to be engaged by centrifuge rotor pins.

11. A method for supporting one or more cell culture devices in the apparatus according to claim 10, the method comprising the steps of:
inserting each cell culture device, of the one or more cell culture devices to be supported, into a slot of the centrifuge bucket; and
hanging the centrifuge bucket on the pins of the centrifuge rotor;
in supporting the one or more cell culture devices.

12. A method for supporting one or more cell culture devices in the apparatus according to claim 10, the method comprising the steps of:
hanging the centrifuge bucket on the pins of the centrifuge rotor; and
inserting each cell culture device, of the one or more cell culture devices to be supported, into a slot of the centrifuge bucket;
in supporting the one or more cell culture devices.

13. An adaptor for use in a centrifuge bucket and a swinging bucket centrifuge rotor, the adaptor comprising: a pair of sidewalls wherein each sidewall comprises a plurality of longitudinally spaced and laterally disposed partitions; wherein each pair of adjacent partitions defines a slot which is dimensioned to receive a cell culture device.

14. The adaptor according to claim 13, wherein the adaptor comprises two sidewalls which, when positioned in parallel spaced apart relation, form a plurality of slots with each slot being dimensioned to receive, and into which may be placed and supported, a cell culture device.

15. The adaptor according to claim 13, wherein the adaptor comprises:
two sidewalls which, when positioned in parallel spaced apart relation, form a plurality of slots with each slot being dimensioned to receive, and into which may be placed and supported, a cell culture device,
a plurality of endwalls, and
a bottom,
in forming an interior chamber for accommodating one or more cell culture devices.

16. The adaptor according to claim 13, further comprising a plurality of handles.

17. A method for supporting one or more cell culture devices in the adaptor according to claim 14, the method comprising the steps of:
inserting the adaptor into a centrifuge bucket; and
inserting each cell culture device, of the one or more cell culture devices to be supported, into a slot of the adaptor;
in supporting the one or more cell culture devices.

18. A method for supporting one or more cell culture devices in the adaptor according to claim 14, the method comprising the steps of:
inserting each cell culture device, of the one or more cell culture devices to be supported, into a slot of the adaptor; and
inserting the adaptor into a centrifuge bucket;
in supporting the one or more cell culture devices.

19. A centrifuge bucket for use in a swinging bucket centrifuge rotor, the centrifuge bucket comprising: a pair of sidewalls wherein each sidewall comprises a plurality of longitudinally spaced and laterally disposed partitions; wherein the pair of sidewalls are positioned in parallel spaced apart relation in forming a plurality of slots with each slot being dimensioned to receive, and into which may be placed and supported, a cell culture device,
a plurality of endwalls, and
a bottom,
in forming an interior chamber for accommodating one or more cell culture devices.

20. The centrifuge bucket according to claim 19, further comprising two sockets disposed on opposite sides of the exterior of centrifuge bucket, and wherein the sockets are dimensioned to be engaged by centrifuge rotor pins.

21. A method for supporting one or more cell culture devices in the centrifuge bucket according to claim 20, the method comprising the steps of:
inserting each cell culture device, of the one or more cell culture devices to be supported, into a slot of the centrifuge bucket; and
hanging the centrifuge bucket on the pins of the centrifuge rotor;
in supporting the one or more cell culture devices.

22. A method for supporting one or more cell culture devices in the apparatus according to claim 20, the method comprising the steps of:
hanging the centrifuge bucket on the pins of the centrifuge rotor; and
inserting each cell culture device, of the one or more cell culture devices to be supported, into a slot of the centrifuge bucket;
in supporting the one or more cell culture devices.

23. A method of centrifuging one or more cell culture devices, the method comprising:
loading the one or more cell culture devices into a centrifuge, wherein each of the one or more cell culture devices is supported in a slot being formed by a pair of adjacent partitions, a slot being dimensioned to receive a cell culture device, wherein a plurality of slots are formed in an apparatus comprising a centrifuge bucket, and wherein a plurality of centrifuge buckets are mounted on a rotor in the centrifuge;
operating the centrifuge; and
removing the one or more cell culture devices from the centrifuge.

24. The method according to claim 23, wherein the centrifuge bucket comprises:
two sidewalls which, when positioned in parallel spaced apart relation, form a plurality of slots with each slot being dimensioned to receive, and into which may be placed and supported, a cell culture device,
a plurality of endwalls, and
a bottom,
in forming an interior chamber for accommodating one or more cell culture devices.

25. The method according to claim 23, wherein the centrifuge bucket further comprises two sockets disposed on opposite sides of the exterior of centrifuge bucket, and wherein the sockets are dimensioned to be engaged by centrifuge rotor pins.

26. The method according to claim 23, wherein the centrifuge bucket further comprises an adaptor inserted within the centrifuge bucket, wherein the adaptor comprises: a pair of sidewalls wherein each sidewall comprises a plurality of longitudinally spaced and laterally disposed partitions; wherein each pair of adjacent partitions defines a slot which is dimensioned to receive a cell culture device.

27. The method according to claim 26, wherein the adaptor comprises two sidewalls which, when positioned in parallel spaced apart relation, form a plurality of slots with each slot being dimensioned to receive, and into which may be placed and supported, a cell culture device.

28. The method according to claim 26, wherein the adaptor comprises:
two sidewalls which, when positioned in parallel spaced apart relation, form a plurality of slots with each slot being dimensioned to receive, and into which may be placed and supported, a cell culture device,
a plurality of endwalls, and
a bottom,
in forming an interior chamber for accommodating one or more cell culture devices.

29. The method according to claim 26, wherein the adaptor further comprises a plurality of handles.

30. A method for separating one or more components of a cell culture within a cell culture device, the method comprising:
loading the cell culture device into a centrifuge, wherein the cell culture device is supported in a slot being formed by a pair of adjacent partitions, a slot being dimensioned to receive the cell culture device, wherein a plurality of slots are formed in an apparatus comprising a centrifuge bucket, and wherein a plurality of centrifuge buckets are mounted on a rotor in the centrifuge;
operating the centrifuge;
removing the cell culture device from the centrifuge; and
aspirating a component comprising cell culture medium from the cell culture device.

31. The method according to claim 30, further comprising introducing a fluid into the cell culture apparatus after the cell culture medium has been aspirated from the cell culture device, contacting and mixing the fluid with pelleted cells contained within the cell culture device in resuspending the cells, and aspirating a component comprising the resuspended cells from the cell culture device.

32. The method according to claim 30, wherein the centrifuge bucket comprises:
two sidewalls which, when positioned in parallel spaced apart relation, form a plurality of slots with each slot being dimensioned to receive, and into which may be placed and supported, a cell culture device,
a plurality of endwalls, and
a bottom,
in forming an interior chamber for accommodating the cell culture device.

33. The method according to claim 30, wherein the centrifuge bucket further comprises two sockets disposed on opposite sides of the exterior of centrifuge bucket, and wherein the sockets are dimensioned to be engaged by centrifuge rotor pins.

34. The method according to claim 30, wherein the centrifuge bucket further comprises an adaptor inserted within the centrifuge bucket, wherein the adaptor comprises: a pair of sidewalls wherein each sidewall comprises a plurality of longitudinally spaced and laterally disposed partitions; wherein each pair of adjacent partitions defines a slot which is dimensioned to receive a cell culture device.

35. The method according to claim 34, wherein the adaptor comprises two sidewalls which, when positioned in parallel spaced apart relation, form a plurality of slots with each slot being dimensioned to receive, and into which may be placed and supported, a cell culture device.

36. The method according to claim 34, wherein the adaptor comprises:
two sidewalls which, when positioned in parallel spaced apart relation, form a plurality of slots with each slot being dimensioned to receive, and into which may be placed and supported, a cell culture device,
a plurality of endwalls, and
a bottom,
in forming an interior chamber for accommodating the cell culture devices.

37. The method according to claim 34, wherein the adaptor further comprises a plurality of handles.

38. A method for harvesting one or more components of a cell culture comprising anchorage-dependent cells within a cell culture device, wherein the cell culture device comprises at least one gas permeable, liquid impermeable membrane that comprises a surface to which anchorage-dependent cells adhere, the method comprising:
loading the cell culture device into a centrifuge, wherein the cell culture device is supported in a slot being formed by a pair of adjacent partitions, a slot being dimensioned to receive the cell culture device, wherein a plurality of slots are formed in an apparatus comprising a centrifuge bucket, and wherein a plurality of centrifuge buckets are mounted on a rotor in the centrifuge;
operating the centrifuge;
removing the cell culture device from the centrifuge; and
removing the one or more components from the cell culture device in harvesting the one or more components.

39. The method according to claim 38, wherein the centrifuge bucket comprises:
two sidewalls which, when positioned in parallel spaced apart relation, form a plurality of slots with each slot being dimensioned to receive, and into which may be placed and supported, a cell culture device,
a plurality of endwalls, and
a bottom,
in forming an interior chamber for accommodating the cell culture device.

40. The method according to claim 38, wherein the centrifuge bucket further comprises two sockets disposed on opposite sides of the exterior of centrifuge bucket, and wherein the sockets are dimensioned to be engaged by centrifuge rotor pins.

41. The method according to claim 38, wherein the centrifuge bucket further comprises an adaptor inserted within the centrifuge bucket, wherein the adaptor comprises: a pair of sidewalls wherein each sidewall comprises a plurality of longitudinally spaced and laterally disposed partitions; wherein each pair of adjacent partitions defines a slot which is dimensioned to receive a cell culture device.

42. The method according to claim 41, wherein the adaptor comprises two sidewalls which, when positioned in parallel spaced apart relation, form a plurality of slots with each slot being dimensioned to receive, and into which may be placed and supported, a cell culture device.

43. The method according to claim 41, wherein the adaptor comprises:
two sidewalls which, when positioned in parallel spaced apart relation, form a plurality of slots with each slot being dimensioned to receive, and into which may be placed and supported, a cell culture device,
a plurality of endwalls, and
a bottom,
in forming an interior chamber for accommodating the cell culture devices.

44. The method according to claim 41, wherein the adaptor further comprises a plurality of handles.

45. The method according to claim 38, wherein the one or more components harvested comprises a component comprising cell culture medium; wherein supernatant, comprising the cell culture medium, is present in the centrifuged cell culture device, and wherein the supernatant is removed from the centrifuged cell culture device in harvesting a component comprising cell culture medium.

46. The method according to claim 45, further comprising introducing a fluid into the cell culture device after the supernatant has been removed from the cell culture device; contacting and mixing the fluid with pelleted cells contained within the cell culture device, in forming a suspension of cells; and removing the cell suspension from the cell culture device in harvesting a component comprising anchorage-dependent cells.

47. The method according to claim 38, further comprising pretreating the anchorage-dependent cells before loading the cell culture device into the centrifuge, wherein the pretreatment is selected from the group consisting of contacting the anchorage-dependent cells with a solution comprising a dissociation reagent, distending the membrane surface to which the cells adhere to loosen cell attachment to the membrane surface, and a combination thereof

48. A method for harvesting the anchorage-dependent cells from a cell culture contained within a cell culture device, wherein the cell culture device comprises at least one elastomeric, gas permeable, liquid impermeable membrane that comprises a surface to which anchorage-dependent cells adhere, the method comprising:
injecting a substance selected from the group consisting of air, fluid, and a combination thereof, into the cell culture device, wherein the substance is injected in an amount sufficient to distend the at least one membrane and to cause the anchorage-dependent cells to loosen their attachment from the membrane surface;
withdrawing the substance, from the cell culture device, in an amount sufficient for the at least one membrane to return to a less distended shape;
contacting and mixing a fluid with the loosened cells within the cell culture device in causing the cells to detach from the membrane surface and to form a suspension of cells; and
removing the suspension of cells from the cell culture device in harvesting the anchorage-dependent cells.

49. The method according to claim 48, wherein the step of injecting a substance into the cell culture device and the step of withdrawing the substance from the cell culture device together comprise a two step process, and wherein the method comprises repeating the two step process before harvesting the anchorage-dependent cells.

50. The method according to claim 49, wherein the two step process is repeated a number of times in a range of from about 2 to about 5.

51. The method according to claim 48, wherein injected into the cell culture device, containing a cell culture comprising anchorage-dependent cells, is a substance comprising air.

52. The method according to claim 48, wherein injected into the cell culture device, containing a cell culture comprising anchorage-dependent cells, is a substance comprising a fluid.

53. The method according to claim 52, wherein the fluid further comprises a dissociation reagent.

54. The method according to claim 49, wherein injected into the cell culture device, containing a cell culture comprising anchorage-dependent cells, is a substance comprising air.

55. The method according to claim 49, wherein injected into the cell culture device, containing a cell culture comprising anchorage-dependent cells, is a substance comprising a fluid.

56. The method according to claim 55, wherein the fluid further comprises a dissociation reagent.

57. The method according to claim 48, wherein the fluid used to contact and mix with the loosened cells comprises cell culture medium present as a component of the cell culture.

58. The method according to claim 48, wherein the fluid used to contact and mix with the loosened cells comprises a fluid introduced into the cell culture device.

59. The method according to claim 48, further comprising centrifuging the cell culture device before the suspension of cells is removed from the cell culture device.

60. The method according to claim 49, further comprising centrifuging the cell culture device before the suspension of cells is removed from the cell culture device.
